(19) United States
(12) Patent Application Publication
(10) Pub. No.: US 2006/0223184 A1
(43) Pub. Date: Oct. 5, 2006

(54) Supports useful in incorporating biomolecules into cells and methods of using thereof

(76) Inventors: Anthony G. Frutos, Painted Post, NY (US); Joydeep Lahiri, Painted Post, NY (US); Santona Pal, Painted Post, NY (US); Elizabeth Tran, Painted Post, NY (US); Brian L. Webb, Painted Post, NY (US)

Correspondence Address: CORNING INCORPORATED, SP-TI-3-1, CORNING, NY, 14831, US

(21) Appl. No.: 11/099,904

(22) Filed: Apr. 5, 2005

(51) Int. Cl. C12N 15/88; C12N 15/86

(52) U.S. Cl. 435/456; 435/458

(57) ABSTRACT

Described herein are supports useful in incorporating biomolecules into cells and methods of making and using thereof.

### BACKGROUND

Reverse transfection represents a new technology for high throughput functional genomic screening. Reverse transfection is a method wherein a defined nucleic acid (a nucleic acid of known sequence or source) is introduced into cells in defined areas of a lawn of cells, in which it will be expressed or will itself have an effect on or interact with a cellular component or function. Unlike conventional screening in which one protein target per well is screened against a compound library, reverse transfection allows multiple protein targets to be expressed and screened per well. This is accomplished by printing microarrays of cDNAs encoding for the desired targets within each well. Simultaneous transfection of the arrayed cDNAs generates patches of transfected mammalian cells, each patch overexpressing a unique recombinant protein, that can be screened for any desired gene function using cell-based or biochemical assays. In general, the protocol for producing reverse transfection arrays involves contact printing mixtures of gelatin and different cDNAs in an array format onto an appropriate substrate. The printed array is incubated with a transfection reagent for a short time, then mammalian cells are plated onto the array surface. Within 24 to 48 hours, patches of cells expressing the various arrayed cDNA can be detected.

One drawback of this protocol is that gelatin or a gelatin-like carrier molecule must be used in the printing ink. This viscous protein-containing ink can complicate the printing process by causing clogging of the quill printing pin and non-reproducible printing.

Described herein are supports with hydrogel layers that can enhance the performance of assays such as, for example, transfection assays. The supports described herein improve the reproducibility of the printing protocol. In one aspect, the supports described herein can improve transfection efficiency, cell viability, and cell attachment. Because the supports described herein can be readily modified, the supports are “tunable,” which permits a wider range of cell types and cell lines that can be assayed.

### SUMMARY

Described herein are supports useful in incorporating biomolecules into cells and methods of making and using thereof. The advantages of the materials, methods, and articles described herein will be set forth in part in the description which follows, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below. It will be appreciated that these drawings depict only typical embodiments of the materials, articles, and methods described herein and are therefore not to be considered limiting of their scope.

FIG. 1 shows a schematic for producing CMD-coated slides.

FIG. 2 shows the surface-mediated transfection on a CMD slide.

FIG. 3 shows the effect of CMD molecular weight on attachment of HEK293T cells on a CMD slide.

FIG. 4 shows the effect of CMD concentration during the preparation of CMD/EDA-coated surfaces on transfection efficiency in HEK293T cells.

### DETAILED DESCRIPTION

Before the present materials, articles, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

By “contacting” is meant an instance of exposure by close physical contact of at least one substance to another substance.

The term “attached” as used herein is any chemical interaction between two components or compounds. The type of chemical interaction that can be formed will vary depending upon the starting materials that are selected and reaction conditions. Examples of attachments described herein include, but are not limited to, covalent, electrostatic, ionic, hydrogen, or hydrophobic bonding.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different polymers and biomolecules are disclosed and discussed, each and every combination and permutation of the polymer and biomolecule are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

I. Supports

Described herein are supports useful in incorporating biomolecules into cells. In one aspect, described herein is a support comprising a substrate, a tie layer, a hydrogel layer, at least one biomolecule, and a cell, wherein the tie layer is covalently bonded to the substrate, the hydrogel layer is attached to the tie layer, the biomolecule is not covalently bonded to the hydrogel layer, and the cell is attached to the hydrogel layer.

In one aspect, the tie layer is covalently bonded to the outer surface of the substrate. The term “outer surface” with respect to the substrate is the region of the substrate that is exposed and can be subjected to manipulation. For example, any surface on the substrate that can come into contact with a solvent or reagent upon contact is considered the outer surface of the substrate. The substrates that can be used herein include, but are not limited to, a microplate, a slide, or any other material that can support cell growth. In one aspect, when the substrate is a microplate, the number of wells and well volume will vary depending upon the scale and scope of the analysis. Other examples of substrates useful herein include, but are not limited to, a cell culture surface such as a 384-well microplate, a 96-well microplate, 24-well dish, 8-well dish, 10 cm dish, or a T75 flask.

For optical or electrical detection applications, the substrate can be transparent, impermeable, or reflecting, as well as electrically conducting, semiconducting, or insulating. For biological applications, the substrate material may be either porous or nonporous and may be selected from either organic or inorganic materials.

In one aspect, the substrate comprises a plastic, a polymeric or co-polymeric substance, a ceramic, a glass, a metal, a crystalline material, a noble or semi-noble metal, a metallic or non-metallic oxide, a transition metal, or any combination thereof. Additionally, the substrate can be configured so that it can be placed in any detection device. In one aspect, sensors can be integrated into the bottom/underside of the substrate and used for subsequent detection. These sensors could include, but are not limited to, optical gratings, prisms, electrodes, and quartz crystal microbalances. Detection methods could include fluorescence, phosphorescence, chemiluminescence, refractive index, mass, electrochemical. In one aspect, the substrate is a resonant waveguide grating sensor.

In one aspect, the substrate can be composed of an inorganic material. Examples of inorganic substrate materials include, but are not limited to, metals, semiconductor materials, glass, and ceramic materials. Examples of metals that can be used as substrate materials include, but are not limited to, gold, platinum, nickel, palladium, aluminum, chromium, steel, and gallium arsenide. Semiconductor materials used for the substrate material include, but are not limited to, silicon and germanium. Glass and ceramic materials used for the substrate material can include, but are not limited to, quartz, glass, porcelain, alkaline earth aluminoborosilicate glass and other mixed oxides. Further examples of inorganic substrate materials include graphite, zinc selenide, mica, silica, lithium niobate, and inorganic single crystal materials.

In another aspect, the substrate comprises a porous, inorganic layer. Any of the porous substrates and methods of making such substrates disclosed in U.S. Pat. No. 6,750,023, which is incorporated by reference in its entirety, can be used herein. In one aspect, the inorganic layer on the substrate comprises a glass or metal oxide. In another aspect, the inorganic layer comprises a silicate, an aluminosilicate, a boroaluminosilicate, a borosilicate glass, or a combination thereof. In a further aspect, the inorganic layer comprises TiO₂, SiO₂, Al₂O₃, Cr₂O₃, CuO, ZnO, Ta₂O₅, Nb₂O₅, ZnO₂, or a combination thereof.

In another aspect, the substrate can be composed of an organic material. Organic materials useful herein can be made from polymeric materials due to their dimensional stability and resistance to solvents. Examples of organic substrate materials include, but are not limited to, polyesters, such as polyethylene terephthalate and polybutylene terephthalate; polyvinylchloride; polyvinylidene fluoride; polytetrafluoroethylene; polycarbonate; polyamide; poly(meth)acrylate; polystyrene, polyethylene; or ethylene/vinyl acetate copolymer.

In one aspect, the substrates described herein have a tie layer covalently bonded to the substrate; however, it is also contemplated that a different tie layer compound can be attached to the substrate by other means in combination with a tie layer compound that is covalently bonded to the substrate. For example, one tie layer compound is covalently bonded to the substrate and a second tie layer compound is electrostatically bonded to the substrate. In one aspect, when the tie layer is electrostatically bonded to the substrate, the compound used to make the tie layer is positively charged and the outer surface of the substrate is treated such that a net negative charge exists so that tie layer compound and the outer surface of the substrate form an electrostatic bond.

In one aspect, the outer surface of the substrate can be derivatized so that there are groups capable of forming a covalent bond with the tie layer compound. The tie layer can be directly or indirectly covalently bonded to the substrate. In the case when the tie layer is indirectly bonded to the substrate, a linker possessing groups that can covalently attach to both the substrate and the tie layer compound can be used. Examples of linkers include, but are not limited to, an ether group, a polyether group, a polyamine, or a polythioether. If a linker is not used, and the tie layer compound is covalently bonded to the substrate, this is referred to as direct covalent attachment.

In one aspect, the tie layer is derived from a compound comprising one or more reactive functional groups that can react with a hydrogel. The phrase “derived from” with respect to the tie layer is defined herein as the resulting residue or fragment of the tie layer compound when it is attached to the substrate. The functional groups permit the attachment of the hydrogel to the tie layer. In one aspect, the functional groups of the tie layer compound comprises an amino group, a thiol group, a hydroxyl group, a carboxyl group, an acrylic acid, an organic and inorganic acid, an ester, an anhydride, an aldehyde, an epoxide, their derivatives or salts thereof, or a combination thereof. In one aspect, the tie layer is derived from a straight or branched-chain aminosilane, aminoalkoxysilane, aminoalkylsilane, aminoarylsilane, aminoaryloxysilane, or a derivative or salt thereof. In a further aspect, the tie layer is derived from 3-aminopropyl trimethoxysilane, N-(beta-aminoethyl)-3-aminopropyl trimethoxysilane, N-(beta-aminoethyl)-3-aminopropyl triethoxysilane, N′-(beta-aminoethyl)-3-aminopropyl methoxysilane, or aminopropylsilsesquixoxane.

In another aspect, the tie layer can be derived from a polymer that is covalently bonded to the substrate. In one aspect, the polymer comprises at least one group capable of forming a covalent bond with the substrate. Examples of such groups include, but are not limited to, an ester group, a carboxylic acid group, an epoxide group, an aldehyde group, or an anhydride group. Examples of polymers that can be used to form the tie layer include, but are not limited to, poly(vinyl acetate-maleic anhydride), poly(styrene-co-maleic anhydride), poly(isobutylene-alt-maleic anhydride), poly(maleic anhydride-alt-1-octadecene), poly(maleic anhydride-alt-1-tetradecene), poly(maleic anhydride-alt-methyl vinyl ether), poly(triethyleneglycol methyvinyl ether-co-maleic anhydride), or poly(ethylene-alt-maleic anhydride).

The hydrogel can be covalently or non-covalently bonded to the tie layer. It is also contemplated that a portion of the hydrogel is covalently bonded to the tie layer and a portion of the hydrogel is not covalently to the tie layer. Thus, for example, a section of the hydrogel can be covalently bonded to the tie layer while another section is not attached at all to the tie layer or, at most, attached so that there is a weak interaction. In one aspect, the hydrogel layer is attached to the tie layer by an electrostatic bond.

Hydrogels are generally polymers that swell upon contact with water. Examples of hydrogels useful herein include, but are not limited to, aminodextran, dextran, DEAE-dextran, chondroitin sulfate, dermatan, heparan, heparin, chitosan, polyethyleneimine, polylysine, dermatan sulfate, heparan sulfate, alginic acid, pectin, carboxymethylcellulose, hyaluronic acid, agarose, carrageenan, starch, polyvinyl alcohol, cellulose, polyacrylic acid, polyacrylamide, polyethylene glycol, or the salt or ester thereof, or a mixture thereof.

The hydrogel can be used as-is or further modified depending upon the desired use of the support. For example, the hydrogel can be modified with one or more different groups so that the hydrogel forms a covalent bond with the tie layer. In one aspect, if the tie layer has an amino group, it can react with one or more groups on the hydrogel to form a covalent or non-covalent bond. For example, the amino group on the tie layer can react with a carboxymethyl-derivatized hydrogel such as carboxymethyl dextran to produce a new covalent bond. Techniques for attaching the hydrogel to the tie layer are described herein.

In one aspect, the hydrogel layer can possess one or more groups that can form covalent and/or non-covalent attachments to the biomolecule. For example, the hydrogel layer comprises one or more cationic groups or one or more groups that can be converted to a cationic group. Examples of such groups include, but are not limited to, substituted or unsubstituted amino groups. In one aspect, when the hydrogel possesses cationic groups, the hydrogel can attach to biomolecules that possess negatively-charged groups to form electrostatic interactions. Conversely, the hydrogel can possess groups that can be converted to anionic groups, wherein the hydrogel can electrostatically attach to positively-charged biomolecules. Finally, the hydrogel can possess one or more groups capable of forming covalent bonds with the biomolecule. Thus, it is contemplated that the hydrogel can form covalent and/or non-covalent bonds with the biomolecule.

The molecular weight of the polymer used to prepare the hydrogel can vary depending upon the selection of the polymer and the biomolecule, the support to be coated, and the cells to be transfected. In one aspect, the polymer has a molecular weight of from 5,000 Da to 2,000,000 Da. In another aspect, the molecular weight of the polymer is 5,000; 10,000; 20,000; 30,000; 40,000; 50,000; 75,000; 100,000; 200,000; 250,000; 300,000; 350,000; 400,000; 450,000; 500,000; 550,000; 600,000; 650,000; 700,000; 750,000; 800,000; 850,000; 900,000; 950,000; 1,000,000; 1,500,000; or 2,000,000 Da, where any value can form a lower or upper endpoint of a molecular weight range. In one aspect, the hydrogel comprises carboxymethyl dextran having a molecular weight of from 5,000 Da to 100,000 Da, 5,000 Da to 90,000 Da; 10,000 Da to 90,000 Da; 20,000 Da to 90,000 Da; 30,000 Da to 90,000 Da; 40,000 Da to 90,000 Da; 50,000 Da to 90,000 Da; or 60,000 Da to 90,000 Da.

The term “biomolecule” is any molecule that is to be introduced or incorporated into a cell. The biomolecule can be covalently or non-covalently bonded to the hydrogel layer. Described below are different methods for using either of these supports.

Examples of biomolecules useful herein include, but are not limited to, a nucleic acid molecule, an antibody, a peptide, a small molecule, a lectin, a modified polysaccharide, a synthetic composite macromolecule, a functionalized nanostructure, a synthetic polymer, a modified/blocked nucleotides/nucleoside, a modified/blocked amino acid, a fluorophore, a chromophore, a ligand, a chelate, an aptamer, or a hapten.

In one aspect, the biomolecule comprises a drug such as a small molecule. In one aspect, any drug that interacts with DNA inside a cell (e.g., induce supercoiling or condensation) can be used. Examples of drugs useful herein include, but are not limited to, spermine, spermidine, or poly-lysine.

In one aspect, the biomolecule can be a protein. For example, the protein can include peptides, fragments of proteins or peptides, membrane-bound proteins, or nuclear proteins. The protein can be of any length, and can include one or more amino acids or variants thereof. The protein(s) can be fragmented, such as by protease digestion, prior to analysis. A protein sample to be analyzed can also be subjected to fractionation or separation to reduce the complexity of the samples. Fragmentation and fractionation can also be used together in the same assay. Such fragmentation and fractionation can simplify and extend the analysis of the proteins.

In another aspect, the biomolecule is a virus. Examples of viruses include, but are not limited to, Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian Immunodeficiency virus, Human immunodeficiency virus type-1, Vaccinia virus, SARS virus, Human immunodeficiency virus type-2, lentivirus, baculovirus, adeno-associated virus, or any strain or variant thereof.

In one aspect, the biomolecule comprises a nucleic acid. The nucleic acid can be an oligonucleotide, deoxyribonucleic acid (DNA), ribonucleic acid (RNA), or peptide nucleic acid (PNA). The nucleic acid of interest introduced by the present method can be nucleic acid from any source, such as a nucleic acid obtained from cells in which it occurs in nature, recombinantly produced nucleic acid, or chemically synthesized nucleic acid. For example, the nucleic acid can be cDNA or genomic DNA or DNA synthesized to have the nucleotide sequence corresponding to that of naturally-occurring DNA. The nucleic acid can also be a mutated or altered form of nucleic acid (e.g., DNA that differs from a naturally occurring DNA by an alteration, deletion, substitution or addition of at least one nucleic acid residue) or nucleic acid that does not occur in nature.

In one aspect, the nucleic acid can be present in a vector such as an expression vector (e.g., a plasmid or viral-based vector). In another aspect, the nucleic acid selected can be introduced into cells in such a manner that it becomes integrated into genomic DNA and is expressed or remains extrachromosomal (i.e., is expressed episomally). In another aspect, the vector is a chromosomally integrated vector. The nucleic acids useful herein can be linear or circular and can be of any size. In one aspect, the nucleic acid can be single or double stranded DNA or RNA.

In one aspect, the nucleic acid can be a functional nucleic acid. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acids can be a small gene fragment that encodes dominant-acting synthetic genetic elements (SGEs), e.g., molecules that interfere with the function of genes from which they are derived (antagonists) or that are dominant constitutively active fragments (agonists) of such genes. SGEs can include, but are not limited to, polypeptides, inhibitory antisense RNA molecules, ribozymes, nucleic acid decoys, and small peptides. The small gene fragments and SGE libraries disclosed in U.S. Patent Publication No. 2003/0228601, which is incorporated by reference, can be used herein.

The functional nucleic acids of the present method can function to inhibit the function of an endogenous gene at the level of nucleic acids, e.g., by an antisense or decoy mechanism, or by encoding a polypeptide that is inhibitory through a mechanism of interference at the protein level, e.g., a dominant negative fragment of the native protein. Alternatively, certain functional nucleic acids can function to potentiate (including mimicking) the function of an endogenous gene by encoding a polypeptide which retains at least a portion of the bioactivity of the corresponding endogenous gene, and may in particular instances be constitutively active.

Functional nucleic acid molecules can interact with any macromolecule, such as DNA, RNA, polypeptides, or carbohydrate chains. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (kd) less than or equal to 10-6, 10-8, 10⁻¹⁰, or 10-12. A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with kds from the target molecule of less than 10-12 M. It is preferred that the aptamers bind the target molecule with a kd less than 10-6, 10-8, 10-10, or 10-12. Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a kd with the target molecule at least 10, 100, 1000, 10,000, or 100,000 fold lower than the kd with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acids. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a kd less than 10-6, 10-8, 10-10, or 10-12. Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSS) are molecules that bind a target nucleic acid molecule forming a complex, and this complex is recognized by RNase P, which cleaves the target molecule. EGSs can be designed to specifically target an RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules can be found in the following non-limiting list of U.S. Pat. Nos. 5,168,053, 5,624,824, 5,683,873, 5,728,521, 5,869,248, and 5,877,162.

It is also understood that the disclosed nucleic acids can be RNA (e.g., for RNA interference (RNAi)). It is thought that RNAi involves a two-step mechanism for RNA interference: an initiation step and an effector step. For example, in the first step, input double-stranded (ds) RNA (siRNA) is processed into small fragments, such as 21-23-nucleotide ‘guide sequences’. RNA amplification appears to be able to occur in whole animals. Typically then, the guide RNAs can be incorporated into a protein RNA complex which is cable of degrading RNA, the nuclease complex, which has been called the RNA-induced silencing complex (RISC). This RISC complex acts in the second effector step to destroy mRNAs that are recognized by the guide RNAs through base-pairing interactions. RNAi involves the introduction by any means of double stranded RNA into the cell which triggers events that cause the degradation of a target RNA. RNAi is a form of post-transcriptional gene silencing.

Disclosed are RNA hairpins that can act in RNAi. In one aspect, the RNAi agent can be small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. When the RNAi agent is a duplex structure of two distinct ribonucleic acids hybridized to each other, e.g., an siRNA, such as d-siRNA, the length of the duplex structure typically ranges from about 15 to 30 bp, usually from about 15 to 29 bp, where lengths between about 20 and 29 bps, e.g., 21 bp, 22 bp, can be used. Where the RNAi agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides. The weight of the RNAi agents of this embodiment typically ranges from about 5,000 daltons to about 35,000 daltons, and in many embodiments is at least about 10,000 daltons and less than about 27,500 daltons, often less than about 25,000 daltons.

In certain aspects, instead of the RNAi agent being an interfering ribonucleic acid, e.g., an siRNA or shRNA as described above, the RNAi agent can encode an interfering ribonucleic acid, e.g., an shRNA, as described above. In other words, the RNAi agent can be a transcriptional template of the interfering ribonucleic acid. In these aspects, the transcriptional template can be a DNA that encodes the interfering ribonucleic acid.

RNAi has been shown to work in a number of cells, including mammalian cells. For work in mammalian cells it is preferred that the RNA molecules which will be used as targeting sequences within the RISC complex are shorter. For example, less than or equal to 50 or 40 or 30 or 29, 28, 27, 26, 25, 24, 23, 22, 21, 20, 19, 18, 17, 16, 15, 14, 13, 12, 11, or 10 nucleotides in length. These RNA molecules can also have overhangs on the 3′ or 5′ ends relative to the target RNA which is to be cleaved. These overhangs can be at least or less than or equal to 1, 2, 3, 4, 5, 6, 7, 8, 9, 10, 15, or 20 nucleotides long. RNAi works in mammalian stem cells, such as mouse ES cells.

For description of making and using RNAi molecules see See, e.g., Hammond et al., Nature Rev Gen 2: 110-119 (2001); Sharp, Genes Dev 15: 485-490 (2001), Waterhouse et al., Proc. Natl. Acad. Sci. USA 95(23): 13959-13964 (1998) all of which are incorporated herein by reference in their entireties and at least form material related to delivery and making of RNAi molecules. The RNAi agents disclosed in U.S. Published Application No. 2003/0228601 and International Publication No. WO2004/0798950, which are incorporated by reference with respect to the different RNAi agents, can also be used herein.

In one aspect, the support further comprises an enhancer molecule. An enhancer molecule is any molecule that can improve cell attachment or adhesion to the hydrogel, transfection efficiency, or cell viability and proliferation. The enhancer molecule can be added to the hydrogel prior to and/or after attachment of hydrogel to the tie layer. Alternatively, the hydrogel can be modified with the enhancer molecule (e.g., chemically react the enhancer molecule with hydrogel and attach the two to one another). The enhancer molecule can be the same or different as the biomolecule. Thus, any of the biomolecules described herein can be used as an enhancer molecule. In one aspect, the enhancer molecule comprises a protein. In another aspect, the enhancer molecule comprises the peptide sequence RGD.

In another aspect, the enhancer molecule such as, for example, RGD, can be conjugated to a polymer that can form a hydrogel. For example, RGD that is conjugated to polyethyleneimine and other products commercially available from Polyplus Transfection can be used as the enhancer molecule. The covalent attachment of RGD peptides to material surfaces allows for cell adhesion to biomaterials of interest. Examples of RGD peptides are known in the art, and include, but are not limited to, head-to-tail cyclic pentapeptides, bicyclic peptides such as H-Glu[cyclo (Arg-Gly-Asp-D-Phe-Lys)]2, and cyclo(Arg-Gly-Asp-D-Phe-Lys). Methods of using RGD peptides can be found in U.S. Pat. Nos. 6,579,322, 6,316,412, 5,955,572, and 5,843,774, all herein incorporated by reference in their entirety for their teaching regarding RGD peptides.

In one aspect, wherein the tie layer is covalently bonded to the substrate, the hydrogel layer is covalently bonded to the tie layer, and the biomolecule is not covalently bonded to the hydrogel layer. In another aspect, the substrate is glass, the tie layer is derived from an aminoalkoxysilane, the hydrogel layer is derived from positively-charged dextran, and the biomolecule comprises a nucleic acid, wherein the aminoalkoxysilane is covalently bonded to the glass, the positively-charged dextran is covalently bonded to the aminoalkoxysilane, and the nucleic acid is electrostatically bonded to the positively-charged dextran. In this aspect, the positively-charged dextran can be aminodextran.

II. Methods for Preparing the Supports

Described herein are methods for producing a substrate comprising (1) covalently attaching a tie layer compound to a substrate; (2) attaching a hydrogel to the tie layer; and (3) attaching a biomolecule to the hydrogel layer. The tie layer, hydrogel, and biomolecule can be attached to one another in any order. For example, the methods described herein contemplate the sequential attachment of the tie layer to the substrate followed by attaching the hydrogel to the tie layer, and the attachment of the biomolecule to the hydrogel. Alternatively, it is contemplated to attach the biomolecule to the hydrogel followed by attaching the hydrogel/biomolecule complex to the tie layer.

The tie layer can be covalently attached to any of the substrates described herein using techniques known in the art. For example, the substrate can be dipped in a solution of the tie compound. In another aspect, the tie compound can be sprayed, vapor deposited, screen printed, or robotically pin printed or stamped on the substrate. This could be done either on a fully assembled substrate or on a bottom insert (e.g., prior to attachment of the bottom insert to a holey plate to form a microplate). In one aspect, after the tie layer is attached to the substrate, the hydrogel can be attached to the layer using similar techniques described above. The enhancer molecule can be added to the hydrogel prior to or after attachment to the tie layer. Alternatively, it can be added prior to or after the attachment of the biomolecule to the hydrogel. Once the tie layer and hydrogel have been attached to the substrate, one or more biomolecules can be attached to the hydrogel using the techniques presented above. In one aspect, when the biomolecule is a nucleic acid, the nucleic acid can be printed on the hydrogel using techniques known in the art. The amount of biomolecule that can be attached to the polymer layer can vary depending upon, for example, the biomolecule selected and the type of cells to be transfected. In one aspect, one or more different biomolecules can be placed at different locations on the support. In the case when different biomolecules are used, the biomolecules can be printed at the same time or different time. It is contemplated that the biomolecule can be contained in a solvent or ink containing carrier molecules such as, for example, agar, collagen, gelatin, alginate gel, starch derivative, dextran, or other protein material that is not cytotoxic for cells. However, these components are optional and can be used as needed. In one aspect, the ink does not contain a carrier molecule such as, for example, gelatin. The techniques disclosed in U.S. Pat. No. 6,652,878 for placing biomolecules on a transfection device, which is incorporated by reference in its entirety, can be used herein.

III. Methods of Use

Described herein are methods for incorporating a biomolecule into a cell. In one aspect, the method comprises contacting the cell with a support comprising a substrate, a tie layer, a hydrogel layer, at least one biomolecule, and a cell, wherein the tie layer is covalently bonded to the substrate, the hydrogel layer is attached to the tie layer, the biomolecule is not covalently bonded to the hydrogel layer, and the cell is attached to the hydrogel layer.

Any of the substrates described herein with one or more biomolecules attached thereto can be used to incorporate a biomolecule into a cell. The term “incorporate” as used herein includes any mechanism that permits the passage of the biomolecule into a cell. For example, any mechanism that permits the passage of the biomolecule or a portion thereof through the cell membrane of a cell is contemplated. Other mechanisms of incorporation include, but are not limited to, endocytosis of the biomolecule by the cell, infection of a cell, (e.g., infection of a cell by a virus), or by transfection of the biomolecule (e.g., a nucleic acid) into the cell. Methods for incorporating biomolecules into cells are well known to those of skill in the art. In one aspect, the cells and a cell growth media are added onto the surface of the support. In one aspect, the cells and cell growth media are added until they cover the top surface of the support.

In one aspect, the cell is a eukaryotic cell. In one aspect, the eukaryotic cell is a mammalian cell (e.g., human, monkey, canine, feline, bovine, or murine), a bacterial cell (or other prokaryotic cells), an insect cell, or a plant cell. Examples of mammalian cells include, but are not limited to, 10.1 mouse fibroblasts, 13-5-1 Chinese hamster ovary epithelial, 132-d5 human fetal fibroblasts; HEK-293 human epithelial kidney; 3T3 or 3T3 NIH or 3T3 Swiss or 3T3-LI mouse embryo fibroblast; BALB/3T3 mouse embryo fibroblast; BHK-21 baby hamster kidney fibroblasts; BS-C-1 monkey kidney epithelial; C2 rat liver epithelial, C2C12 mouse muscle fibroblast, C2H mouse embryo fibroblast; C4, C6 Caco-2 human adenocarcinoma epithelial cells, CHO or CHO-7 or CHO-IR or CHO-K1 or CHO-K2 or CHO-T or CHO Dhfr −/−Chinese hamster ovary epithelial; COS or COS-1 or COS-6 or COS-7 or COS-M6A African green monkey kidney, SV40 transformed fibroblast; HeLa or HeLa B or HeLa T4 human cervix carcinoma epithelial; Hep G2 human hepatoblastoma epithelial; MDCK (NBL-2) canine kidney epithelial; MEF mouse embryo fibroblast; MRC-5; NRK or NRK-52E normal rat epithelial etc. In one aspect, the cells can be plated at a density of 0.3×10⁵/cm² to 3.0×10⁵/cm², 0.5×10⁵/cm² to 2.0×10⁵/cm², or 0.5×10⁵/cm² to 1.0×10⁵/cm².

In one aspect, a transfection agent can be used. The transfection agent facilitates the incorporation of the biomolecule into the cells that are on the surface of the support. In one aspect, the support is incubated with transfection agent prior to contacting the support with the cells. In another aspect, the transfection agent can be incubated with the biomolecule prior to depositing on the hydrogel surface. In one aspect, the transfection agent comprises a cationic lipid or a cationic liposome. Other transfection agents useful herein include, but are not limited to, (1) DOTAP™, a monocationic compound liposome formulation; (2) DOSPER™, a liposomal formulation of a polycationic compound; (3) Fugene 6™, a non-liposomal blend of lipids and other compounds; (4) X-tremeGENE Q2 Transfection Reagent for HeLa, Jurkat and K-562 cell types; (5) SuperFect™, an activated dendrimer (6) Efectene, a cationic non-liposomal lipids formulation; and 97) CLONfectin™ a cationic, amphiphilic lipid. In another aspect, the transfection agent comprises Effectine, Lipofectamine, Transfast, calcium phosphate, DEAE-dextran, or polyethyleneimine. In other aspects, transfection agents commercially available from Promega, Qiagen, and Invitrogen can be used herein.

The type of attachment between the biomolecule and the hydrogel can influence transfection parameters. In one aspect, when the biomolecule is not covalently bonded to the hydrogel layer the entire biomolecule or a portion thereof can be incorporated into the cell. In another aspect, when the biomolecule is covalently bonded to the hydrogel layer, after the cells are attached to the support, the covalent bond between the biomolecule and hydrogel layer can be cleaved using techniques known in the art (e.g., proteases, nucleases, restriction enzymes, photocleaving agents) followed by incorporation of the biomolecule into the cell. Additionally, the covalent bond between the tie layer and the hydrogel could be cleaved using techniques known in the art (e.g., proteases, nucleases, restriction enzymes, photocleaving agents), allowing the hydrogel and the biomolecule to be taken up together by the cell. Alternatively, it is possible for a portion of the hydrogel that is covalently bonded to the biomolecule to be incorporated into the cell with the biomolecule. In this aspect, a section of the hydrogel is not attached to the tie layer and is free to be incorporated into the cell.

The efficiency of the transfection can be monitored using direct or indirect assay methods. For example, the cells can incorporate a reporter gene which is used to confirm the protein expression of the biomolecules. Common reporter genes include, for example, green fluorescent protein (GFP), chloramphenical acetyl transferase for a CAT ELISA immunological assay, firefly luciferase, β-galactosidase, or human growth hormone (hGH).

In one aspect, described herein is a method for detecting the activity of a biomolecule, comprising (a) contacting a support comprising a substrate, a tie layer, a hydrogel layer, the biomolecule, and cell, wherein the tie layer is covalently bonded to the substrate, the hydrogel layer is attached to the tie layer, the biomolecule is not covalently bonded to the hydrogel layer, and the cell is attached to the hydrogel layer, wherein the biomolecule is incorporated into the cell and modulates a response, and (b) detecting the response.

The term “modulate” is defined herein as the ability of the biomolecule to decrease or increase the activity relative to a control. The “control” can be either the amount of activity in the absence of the biomolecule. The term “activity” means and is meant to include any measurable physical, chemical, or biological affinity between two or more molecules or between two or more moieties on the same or different molecules. As will be understood from the compositions and methods disclosed herein, any measurable interaction between molecules can be involved in and are suitable for the methods and compositions disclosed herein. General examples include interactions between small molecules, between proteins, between nucleic acids, between small molecules and proteins, between small molecules and nucleic acids, between proteins and nucleic acids, and the like.

Examples of activities that can be involved in and/or determined by the supports and methods disclosed herein include, but are not limited to, an attraction, affinity, a binding specificity, an electrostatic interaction, a van der Waals interaction, a hydrogen bonding interaction, and the like.

One specific type of activity that can be involved in and/or determined by the methods and supports disclosed herein is an interaction between a ligand (e.g., a potential therapeutic agent, a small molecule, an agonist, an antagonist, an inhibitor, an activator, a suppressor, a stimulator, and the like) and a protein (e.g., a receptor, a channel, a signal transducer, an enzyme, and the like). For example, an interaction between a potential therapeutic agent and a target protein can indicate a potential therapeutic activity for the agent. In another example, an interaction between a small molecule (e.g., a lipid, a carbohydrate, etc.) and an enzyme (e.g., a kinase, a phosphatase, a reductase, an oxidase, and the like) can indicate enzymatic activity or substrate specificity.

In another example of a type of activity that can be involved in and/or determined by the methods and supports disclosed herein is an interaction between two proteins or fragments thereof (e.g., an enzyme and a protein substrate or an antibody and an antigen or an epitope of an antigen). An example of this interaction can include, but is not limited to, the binding of a kinase, a protease, a phosphatase, and the like to a substrate protein. Such interactions can, but need not, result in a reaction or chemical transformation (e.g., phosphorylation, cleavage, or dephosphorylation). Another example of an interaction includes the binding or affinity of an antibody for an antigen or epitope of an antigen.

Yet another type of activity that can be involved in and/or detected by the compositions and methods disclosed herein includes an interaction between a protein (e.g., a polymerase, endonuclease, or ligase) and a nucleic acid.

In one aspect, the supports and methods described herein can measure the activity of a transfected nucleic acid (e.g., RNA). The techniques disclosed in U.S. Published Application No. 2003/0228601 to Sabatini and International Publication No. WO 2004/078950 to Chi et al. for transfecting nucleic acids such as, for example, interfering RNA, can be used herein.

In one aspect, an array can be used in any of the methods described herein. In one aspect, the array comprises a plurality of biomolecules on the substrate, wherein the biomolecules are on discrete and defined locations on the support. Arrays have been used for a wide range of applications such as gene discovery, disease diagnosis, drug discovery (pharmacogenomics) and toxicological research (toxicogenomics). An array is an orderly arrangement of biomolecules. The typical method involves contacting an array of biomolecules with a target of interest to identify those compounds in the array that bind to the target. Arrays are generally described as macro-arrays or micro-arrays, the difference being the size of the sample spots. Macro-arrays contain sample spot sizes of about 300 microns or larger whereas micro-arrays are typically less than 200 microns in diameter and typically contain thousands of spots. In one aspect, the distance between each biomolecule in the array can be from 200 to 500 μm.

Methods for producing arrays are known in the art. For example, Fodor et al., 1991, Science 251:767-773 describe an in situ method that utilizes photo-protected amino acids and photo lithographic masking strategies to synthesize miniaturized, spatially-addressable arrays of peptides. This in situ method has recently been expanded to the synthesis of miniaturized arrays of oligonucleotides (U.S. Pat. No. 5,744,305). Another in situ synthesis method for making spatially-addressable arrays of immobilized oligonucleotides is described by Southern, 1992, Genomics 13:1008-1017; see also Southern & Maskos, 1993, Nucl. Acids Res. 21:4663-4669; Southern & Maskos, 1992, Nucl. Acids Res. 20:1679-1684; Southern & Maskos, 1992, Nucl. Acids Res. 20:1675-1678. In this method, conventional oligonucleotide synthesis reagents are dispensed onto physically masked glass slides to create the array of immobilized oligonucleotides. U.S. Pat. No. 5,807,522 describes a deposition method for making micro arrays of biological samples that involves dispensing a known volume of reagent at each address of the array by tapping a capillary dispenser on the substrate under conditions effective to draw a defined volume of liquid onto the substrate.

In one aspect, an array of nucleic acid(s) can be printed on any of the substrates described herein. The techniques disclosed in U.S. Published Application No. 2003/0228601 to Sabatini can be used herein, which is incorporated by reference with respect to the different arrays and nucleic acid libraries that can be used in the methods described herein.

In one aspect, a cell transfected with interfering RNA can reduce or prevent the production of a particular protein. Examples of proteins that are useful for drug testing include, but are not limited to: (1) liver enzymes for an ADME and toxicology assay; (2) cytokine, growth factor and hormone receptors e.g. epidermal growth factor receptor (EGF-R), fibroblast growth factor receptor 1 (FGFR-1, FGFR-2, FGFR-3); insulin-like growth factor binding proteins (protein-1, (IGFBP-1/GF-1 complex) protein-1/GF-1 complex, (IGFBP-2) protein-2, IGFB-3, insulin receptor (a receptor protein tyrosine kinase that mediates the activity of insulin; Interleukin receptors (IL-1, sRI, IL-1RacP, IL-2 sRα, IL-2 sRβ, IL-18); leptin receptors; VEGF receptors (R1, flk-1, Flt-4, tie-1, tek/tie-2); androgen receptor, estrogen receptors (ER, ER-β), (3) adrenergic neurotransmitter receptors, (4) other neurotransmitters (Cb₂, D₁, D₂long, D3, D2,4, M1, M2, M3, serotonin receptors (5-HT_{1A}, 5-HT₆, 5-HT₇), nicotinic acetylcholine receptors, muscarinic acetylcholine receptors, (5) calcium channels, (6) angiogenesis regulators, and (7) G proteins and g-protein coupled receptors.

Additional uses for transfecting cells with nucleic acids include, but are not limited to: infer the expression of a gene product by detecting the expression of a co-transfected plasmid encoding a marker protein (e.g. GFP, luciferase, beta-galactosidase, or any protein to which a specific antibody is available), express all the components of a multi-subunit complex (e.g. the T-cell receptor) in the same cells, express all the components of a signal transduction pathway (e.g. MAP kinase pathway) in the same cells, and express all the components of a pathway that synthesizes a small molecule (e.g. polyketide synthetase). In addition, the capacity to co-transfect allows the creation of microarrays with combinatorial combinations of co-expressed plasmids. This capacity is particularly useful for implementing mammalian two-hybrid assays in which plasmids encoding bait and prey proteins are co-transfected into the same cells by spotting them in one feature of the microarray.

Moreover, the capacity to co-transfect is also useful when the goal is to promote differentiation of the transfected cells along a certain tissue lineage. For example, combination of genes can be expressed in a stem or early progenitor cells that will force the differentiation of the cells into endothelial, liver, heart, pancreatic, lymphoid, islet, brain, lung, kidney or other cell types. In this fashion, arrays can be made with primary-like cells that can be used to examine interactions of protein or small molecules that are cell-type specific.

The expression products produced by the transfected cells can be detected by techniques known in the art. For example, the expression product can be detected by immunofluorescence, microscopy, a cell-based assay, enzyme immunocytochemistry, autoradiography, or label-independent detection.

### EXAMPLES

The following examples are put forth so as to provide those of ordinary skill in the art with a complete disclosure and description of how the materials, articles, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

A. Materials

HEK293 cells were obtained from the American Type Cell Culture. Iscove's DMEM media (+Pen/Strep) was supplemented with 10% Fetal bovine serum. Carboxymethyldextran (CMD) was purchased from Fluka (Catalog #27560). 1-[3-(Dimethylamino)propyl]-3-ethylcarbodiimide hydrochloride (EDC) and N-hydroxysuccinimide (NHS) were purchased from Aldrich. Ethylenediamine (EDA) was purchased from Acros. The buffer solution used was 100 mM borate buffer (pH 9).

B. Preparation of Carboxymethyldextran Surfaces

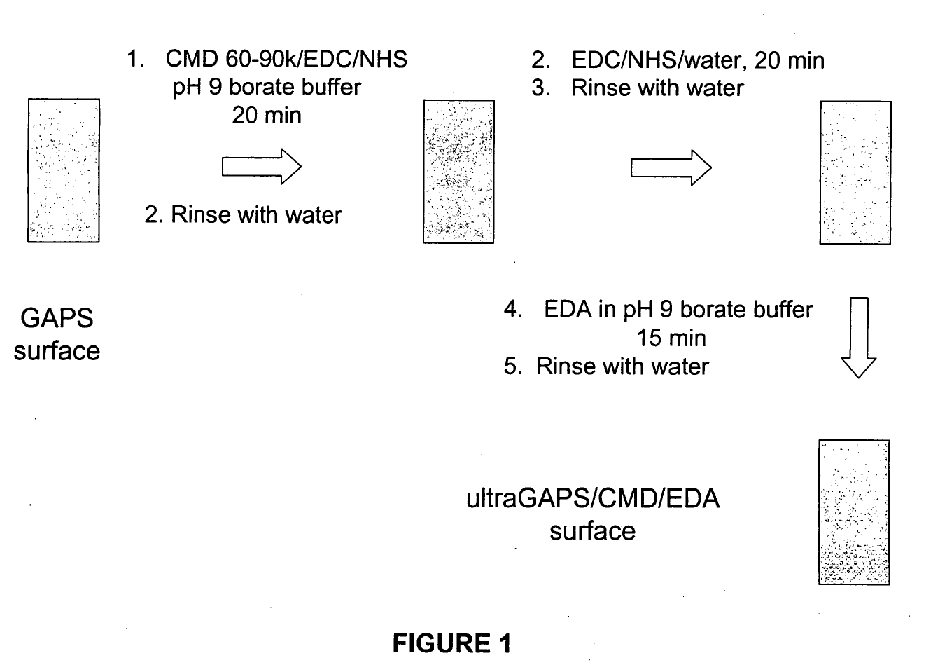

A 20 mL solution containing 10 mg/mL CMD, 38.3 mg/mL EDC, 5.8 mg/mL NHS in Millipore water was prepared. The solution was vortexed for 20 minutes. To this solution, 20 mL of pH 9 borate buffer was added and mixed. This solution was poured over Corning Ultra-GAPS slides in a coplin staining jar. After 20 minutes, each slide was washed with water and dried with a stream of nitrogen. A 50 mL solution containing 14.4 mg/mL EDC and 1.7 mg/mL NHS was prepared. This solution was then poured over the slides. After 20 minutes, each slide was washed with water and dried with a stream of nitrogen. Next, the slides were contacted with a solution of 300 mM (20 uL EDA/mL buffer) EDA in pH 9 borate buffer for 15 minutes. The slides were then rinsed with water and dry with a stream of nitrogen. FIG. 1 provides schematic for producing the slides.

C. Printing of Reverse Transfection Microarrays in the Absence of a Carrier Protein in the Printing Ink

Solutions containing the pQBI25 plasmid DNA, which contains the eGFP gene at the indicated concentrations, were made in a TE buffer solution (10 mM Tris-HCl (pH 8.0), 1 mM EDTA). The solutions were printed in a microarray format onto the CMD slide using a quill pin (Telechem CMP10B) and a Cartesian PixSys 5500 contact pin printer. The printed slides were allowed to dry for 1 hour.

D. Surface-Mediated Transfection on CMD Slide

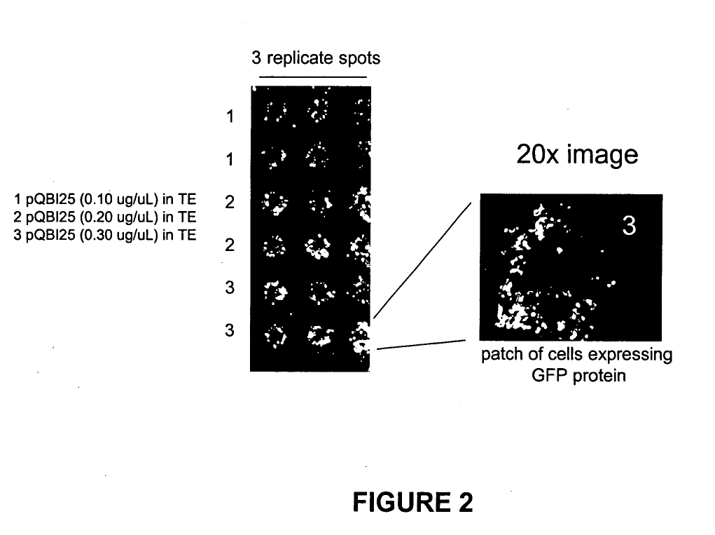

The slide was then treated with Effectene transfection reagent (150 uL EC buffer+16 uL Enhancer+25 uL Effectene) using a Grace BioLabs Cover Chamber (PC200). The Effectene solution was removed and the surface was overlayed with HEK293 cells (5×10⁶ per side contained in a QuadriPerm 4-compartment culture dish). Patches of GFP-expressing cells were observed 24-48 hours after adding the cells. Fluorescent microscope images were taken after 48 hours. FIG. 2 shows the surface mediated transfection on the CMD slide.

E. Effect of CMD Molecular Weight on Attachment of HEK293T Cells to on CMD/EDA-Coated Surfaces

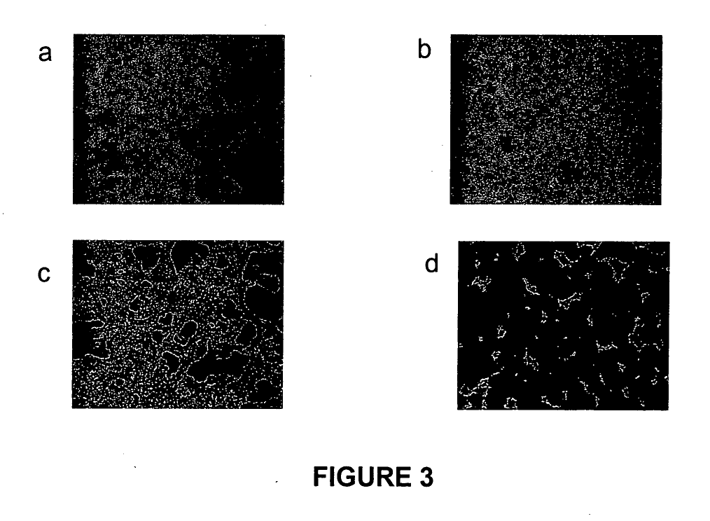

The molecular weight of the CMD used to prepare the CMD surface had an effect on cell attachment. The use of high molecular weight CMD (>500,000 Da) resulted in poor attachment of HEK293T cells, leading to poor transfected patches (FIG. 3d). Lower molecular weight CMD (12,000 Da; 60,000-90,000 Da; and 250,000 Da) gave much better cell attachment and thus improved surface-mediated transfection (FIGS. 3a-c, respectively).

F. Effect of CMD Concentration During Preparation of CMD/EDA-Coated Surfaces on Transfection Efficiency in HEK293T Cells

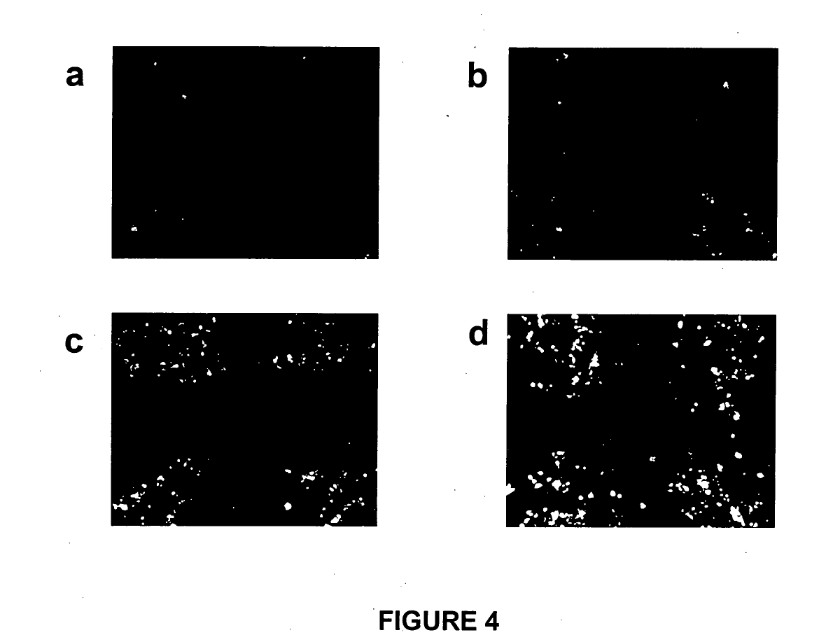

The concentration of CMD used to prepare the CMD slides affected the transfection efficiency. The optimal CMD concentration was 5 mg/mL (FIG. 4d) when compared to 1.25 mg/mL; 2.5 mg/mL; and 3.75 mg/mL (FIGS. 4a-c, respectively).

Throughout this application, various publications are referenced. The disclosures of these publications in their entireties are hereby incorporated by reference into this application in order to more fully describe the compounds, compositions and methods described herein.

Various modifications and variations can be made to the materials, methods, and articles described herein. Other aspects of the materials, methods, and articles described herein will be apparent from consideration of the specification and practice of the materials, methods, and articles disclosed herein. It is intended that the specification and examples be considered as exemplary.

What is claimed:

1. A method for incorporating a biomolecule into a cell, comprising contacting the cell with a support comprising a substrate, a tie layer, a hydrogel layer, at least one biomolecule, and a cell, wherein the tie layer is covalently bonded to the substrate, the hydrogel layer is attached to the tie layer, the biomolecule is not covalently bonded to the hydrogel layer, and the cell is attached to the hydrogel layer.

2. The method of claim 1, wherein the substrate comprises a plastic, a polymeric or co-polymeric substance, a ceramic, a glass, a metal, a crystalline material, a noble or semi-noble metal, a metallic or non-metallic oxide, a transition metal, or any combination thereof.

3. The method of claim 1, wherein the substrate comprises a porous, inorganic layer.

4. The method of claim 3, wherein the inorganic layer comprises a silicate, an aluminosilicate, a boroaluminosilicate, a borosilicate glass, or a combination thereof.

5. The method of claim 3, wherein the inorganic layer comprises TiO₂, SiO₂, Al₂O₃, Cr₂O₃, CuO, ZnO, Ta₂O₅, Nb₂O₅, ZnO₂, or a combination thereof.

6. The method of claim 1, wherein the tie layer is attached to the substrate by a covalent bond.

7. The method of claim 1, wherein the tie layer is derived from a compound comprising one or more functional groups that permit the attachment of the hydrogel to the tie layer.

8. The method of claim 7, wherein the functional group comprises an amino group, a thiol group, a hydroxyl group, a carboxyl group, an acrylic acid, an organic and inorganic acid, an ester, an anhydride, an aldehyde, an epoxide, their derivatives or salts thereof, or a combination thereof.

9. The method of claim 1, wherein the tie layer is derived from a straight or branched-chain aminosilane.

10. The method of claim 9, wherein the aminosilane comprises aminoalkoxysilane, aminoalkylsilane, aminoarylsilane, aminoaryloxysilane, or a derivative or salt thereof.

11. The method of claim 1, wherein the tie layer is derived from N-(beta-aminoethyl)-3-aminopropyl trimethoxysilane, N-(beta-aminoethyl)-3-aminopropyl triethoxysilane, N′-(beta-aminoethyl)-3-aminopropyl methoxysilane, or aminopropylsilsesquixoxane.

12. The method of claim 1, wherein the tie layer is derived from 3-aminopropyl triethoxysilane.

13. The method of claim 1, wherein the tie layer is derived from a polymer having at least one group capable of forming a covalent bond with the substrate.

14. The method of claim 13, wherein the polymer comprises poly(vinyl acetate-maleic anhydride), poly(styrene-co-maleic anhydride), poly(isobutylene-alt-maleic anhydride), poly(maleic anhydride-alt-1-octadecene), poly(maleic anhydride-alt-1-tetradecene), poly(maleic anhydride-alt-methyl vinyl ether), poly(triethyleneglycol methyvinyl ether-co-maleic anhydride), or poly(ethylene-alt-maleic anhydride).

15. The method of claim 1, wherein the hydrogel layer is attached to the tie layer by a covalent bond.

16. The method of claim 1, wherein the hydrogel layer is attached to the tie layer by a non-covalent bond.

17. The support of claim 1, wherein the hydrogel layer is attached to the tie layer by an electrostatic bond.

18. The method of claim 1, wherein the hydrogel layer comprises at least one cationic group or comprises at least one group that can be converted to a cationic group.

19. The method of claim 1, wherein hydrogel layer comprises at least one amino group.

20. The method of claim 1, wherein the hydrogel layer is derived from aminodextran, dextran, DEAE-dextran, chondroitin sulfate, dermatan, heparan, heparin, chitosan, polyethyleneimine, polylysine, dermatan sulfate, heparan sulfate, alginic acid, pectin, carboxymethylcellulose, hyaluronic acid, agarose, carrageenan, starch, polyvinyl alcohol, cellulose, polyacrylic acid, polyacrylamide, polyethylene glycol, or the salt or ester thereof, or a mixture thereof.

21. The method of claim 1, wherein the hydrogel layer is derived from carboxymethyldextran.

22. The method of claim 1, wherein the biomolecule is attached to the hydrogel layer by an electrostatic bond.

23. The method of claim 1, wherein the biomolecule comprises an antibody, a peptide, a small molecule, a lectin, a modified polysaccharide, a synthetic composite macromolecule, a functionalized nanostructure, a synthetic polymer, a modified/blocked nucleotides/nucleoside, a modified/blocked amino acid, a fluorophore, a chromophore, a ligand, a chelate, an aptamer, or a hapten.

24. The method of claim 1, wherein the biomolecule comprises a nucleic acid comprising a ribonucleic acid, a deoxyribonucleic acid, or an oligonucleotide.

25. The method of claim 1, wherein the biomolecule comprises a nucleic acid.

26. The method of claim 25, wherein the nucleic acid inhibits a function of a gene in the cell.

27. The method of claim 1, wherein the biomolecule comprises an oligonucleotide.

28. The method of claim 1, wherein the biomolecule comprises DNA.

29. The method of claim 28, wherein the DNA comprises plasmid DNA.

30. The method of claim 1, wherein the biomolecule comprises RNA.

31. The method of claim 1, wherein the biomolecule comprises a protein.

32. The method of claim 1, wherein the biomolecule comprises a virus.

33. The method of claim 1, wherein the biomolecule comprises an RNAi agent.

34. The method of claim 33, wherein the RNAi agent comprises an interfering ribonucleic acid.

35. The method of claim 33, wherein the RNAi agent comprises an oligoribonucleotide present in a duplex structure or a single ribooligonucleotide.

36. The method of claim 33, wherein the RNAi agent is siRNA.

37. The method of claim 33, wherein the RNAi agent is a transcription template of an interfering ribonucleic acid.

38. The method of claim 37, wherein the transcription template is a transcription template of an interfering ribonucleic acid.

39. The method of claim 38, wherein the transcription template is a deoxyribonucleic acid that encodes an interfering RNA.

40. The method of claim 39, wherein the interfering RNA is shRNA.

41. The method of claim 25, wherein the nucleic acid is contained in a vector.

42. The method of claim 41, wherein the vector is an expression vector.

43. The method of claim 41, wherein the vector is an episomal vector or a chromosomally integrated vector.

44. The method of claim 41, wherein the vector is a plasmid or a viral-based vector.

45. The method of claim 1, wherein a plurality of biomolecules are present on the support, wherein the biomolecules are on discrete and defined locations on the support to produce an array.

46. The method of claim 45, wherein the array comprises at least 96 distinct and defined locations.

47. The method of claim 45, wherein the support comprises at least 192 distinct and defined locations.

48. The method of claim 45, wherein the distinct and defined locations are from 200 to 500 μm apart from each other.

49. The method of claim 1, wherein the biomolecule is a nucleic acid, wherein the nucleic acid encodes a polypeptide that is expressed in the cell.

50. The method of claim 1, wherein the biomolecule is a nucleic acid, wherein the nucleic acid inhibits a function of a gene in the cell upon incorporation of the biomolecule into the cell.

51. The method of claim 1, wherein the support further comprises an enhancer molecule.

52. The method of claim 51, wherein the enhancer molecule comprises an antibody, a peptide, a small molecule, a lectin, a modified polysaccharide, a synthetic composite macromolecule, a functionalized nanostructure, a synthetic polymer, a modified/blocked nucleotides/nucleoside, a modified/blocked amino acid, a fluorophore, a chromophore, a ligand, a chelate, a hapten, a virus, a nucleic acid comprising a ribonucleic acid, a deoxyribonucleic acid, an aptamer, or an oligonucleotide.

53. The method of claim 51, wherein the enhancer molecule comprises a protein.

54. The method of claim 51, wherein the enhancer molecule comprises a RGD peptide.

55. The method of claim 54, wherein the RGD peptide comprises a head-to-tail cyclic pentapeptide, a bicyclic peptide, or a RGD peptide conjugated to a polymer.

56. The method of claim 1, wherein the tie layer is covalently attached to the substrate, the hydrogel layer is covalently attached to the tie layer, and the biomolecule is not covalently attached to the hydrogel layer.

57. The method of claim 1, wherein the substrate is glass, the tie layer is derived from an aminoalkoxysilane, the hydrogel layer is derived from positively-charged dextran, and the biomolecule comprises a nucleic acid, wherein the aminoalkoxysilane is covalently attached to the glass, the positively-charged dextran is covalently attached to the aminoalkoxysilane, and the nucleic acid is electrostatically attached to the positively-charged dextran.

58. The method of claim 1, wherein the support is a slide, a microplate, an array, or a substrate that can support cell growth.

59. The method of claim 1, wherein the cell is a eukaryotic prokaryotic cell.

60. The method of claim 1, wherein the cell is a prokaryotic cell.

61. The method of claim 1, wherein the cell is a mammalian cell.

62. The method of claim 1, wherein the cell is a bacterial cell, an insect cell, or a plant cell.

63. The method of claim 1, wherein after the contacting step, contacting the cells and support with a transfection agent.

64. The method of claim 63, wherein the transfection agent comprises a cationic lipid or a cationic liposome.

65. The method of claim 63, wherein the transfection agent comprises Effectine, Lipofectamine, Transfast, calcium phosphate, DEAE-dextran, or polyethyleneimine.

66. The method of claim 1, wherein the method does not use a carrier molecule.

67. The method of claim 66, wherein the carrier molecule is gelatin.

68. The method of claim 1, wherein during the contacting step, the cells are plated at a density of 0.3×10⁵/cm² to 3.0×10⁵/Cm².

69. The method of claim 1, wherein during the contacting step, the cells are plated at a density of 0.5×10⁵/cm² to 2.0×10⁵/cm².

70. The method of claim 1, wherein during the contacting step, the cells are plated at a density of 0.5×10⁵/cm² to 1.0×10⁵/cm².

71. The method of claim 1, wherein after the contacting step, cleaving the covalent bond between the tie layer and the hydrogel.

72. A method for incorporating a biomolecule into a cell, comprising contacting the cell with a support comprising a substrate, a tie layer, a hydrogel layer, at least one biomolecule, and a cell, wherein the tie layer is covalently bonded to the substrate, the hydrogel layer is attached to the tie layer, the biomolecule is covalently bonded to the hydrogel layer, and the cell is attached to the hydrogel layer.

73. The method of claim 72, wherein after the contacting step, cleaving the covalent bond between the biomolecule and the hydrogel.

74. The method of claim 71, wherein after the contacting step, cleaving the covalent bond between the tie layer and the hydrogel.

75. A method for detecting the activity of a biomolecule, comprising (a) contacting a support comprising a substrate, a tie layer, a hydrogel layer, the biomolecule, and cell, wherein the tie layer is covalently bonded to the substrate, the hydrogel layer is attached to the tie layer, the biomolecule is not covalently bonded to the hydrogel layer, and the cell is attached to the hydrogel layer, wherein the biomolecule is incorporated into the cell and modulates a response, and (b) detecting the response.

76. A support comprising a substrate, a tie layer, a hydrogel layer, at least one biomolecule, and a cell, wherein the tie layer is covalently bonded to the substrate, the hydrogel layer is attached to the tie layer, the biomolecule is not covalently bonded to the hydrogel layer, and the cell is attached to the hydrogel layer.

77. The support of claim 76, wherein the substrate comprises a plastic, a polymeric or co-polymeric substance, a ceramic, a glass, a metal, a crystalline material, a noble or semi-noble metal, a metallic or non-metallic oxide, a transition metal, or any combination thereof.

78. The support of claim 76, wherein the substrate comprises a porous, inorganic layer.

79. The support of claim 78, wherein the inorganic layer comprises a glass or metal oxide.

80. The support of claim 78, wherein the inorganic layer comprises a silicate, an aluminosilicate, a boroaluminosilicate, a borosilicate glass, or a combination thereof.

81. The support of claim 78, wherein the inorganic layer comprises TiO₂, SiO₂, Al₂O₃, Cr₂O₃, CuO, ZnO, Ta₂O₅, Nb₂O₅, ZnO₂, or a combination thereof.

82. The support of claim 76, wherein the tie layer is derived from a compound comprising one or more functional groups that permit the attachment of the hydrogel to the tie layer.

83. The support of claim 82, wherein the functional group comprises an amino group, a thiol group, a hydroxyl group, a carboxyl group, an acrylic acid, an organic and inorganic acid, an ester, an anhydride, an aldehyde, an epoxide, their derivatives or salts thereof, or a combination thereof.

84. The support of claim 76, wherein the tie layer is derived from a straight or branched-chain aminosilane.

85. The support of claim 76, wherein the aminosilane comprises aminoalkoxysilane, aminoalkylsilane, aminoarylsilane, aminoaryloxysilane, or a derivative or salt thereof.

86. The support of claim 76, wherein the tie layer is derived from N-(beta-aminoethyl)-3-aminopropyl trimethoxysilane, N-(beta-aminoethyl)-3-aminopropyl triethoxysilane, N′-(beta-aminoethyl)-3-aminopropyl methoxysilane, or aminopropylsilsesquixoxane.

87. The support of claim 76, wherein the tie layer is derived from 3-aminopropyl triethoxysilane.

88. The support of claim 76, wherein the tie layer is derived from a polymer having at least one group capable of forming a covalent bond with the substrate.

89. The support of claim 88, wherein the polymer comprises poly(vinyl acetate-maleic anhydride), poly(styrene-co-maleic anhydride), poly(isobutylene-alt-maleic anhydride), poly(maleic anhydride-alt-1-octadecene), poly(maleic anhydride-alt-1-tetradecene), poly(maleic anhydride-alt-methyl vinyl ether), poly(triethyleneglycol methyvinyl ether-co-maleic anhydride), or poly(ethylene-alt-maleic anhydride).

90. The support of claim 76, wherein the hydrogel layer is attached to the tie layer by a covalent bond.

91. The support of claim 76, wherein the hydrogel layer is attached to the tie layer by a non-covalent bond.

92. The support of claim 76, wherein the hydrogel layer is attached to the tie layer by an electrostatic bond.

93. The support of claim 76, wherein the hydrogel layer comprises at least one cationic group or comprises at least one group that can be converted to a cationic group.

94. The support of claim 76, wherein hydrogel layer comprises at least one amino group.

95. The support of claim 76, wherein the hydrogel layer is derived from aminodextran, dextran, DEAE-dextran, chondroitin sulfate, dermatan, heparan, heparin, chitosan, polyethyleneimine, polylysine, dermatan sulfate, heparan sulfate, alginic acid, pectin, carboxymethylcellulose, hyaluronic acid, agarose, carrageenan, starch, polyvinyl alcohol, cellulose, polyacrylic acid, polyacrylamide, polyethylene glycol, or the salt or ester thereof, or a mixture thereof.

96. The support of claim 76, wherein the hydrogel layer is derived from dextran.

97. The support of claim 76, wherein the hydrogel layer is derived from carboxymethyl dextran having a molecular weight of from 5,000 Da to 2,000,000 Da.

98. The support of claim 76, wherein the hydrogel layer is derived from carboxymethyl dextran having a molecular weight of from 60,000 Da to 90,000 Da.

99. The support of claim 76, wherein the biomolecule is bonded to the hydrogel layer by an electrostatic bond.

100. The support of claim 76, wherein the biomolecule comprises an antibody, a peptide, a small molecule, a lectin, a modified polysaccharide, a synthetic composite macromolecule, a functionalized nanostructure, a synthetic polymer, a modified/blocked nucleotides/nucleoside, a modified/blocked amino acid, a fluorophore, a chromophore, a ligand, a chelate, an aptamer, or a hapten.

101. The support of claim 76, wherein the biomolecule comprises a nucleic acid comprising an oligonucleotide.

102. The support of claim 76, wherein the biomolecule comprises a nucleic acid comprising a deoxyribonucleic acid.

103. The support of claim 102, wherein the deoxyribonucleic acid comprises plasmid DNA.

104. The support of claim 76, wherein the biomolecule comprises a nucleic acid comprising a ribonucleic acid.

105. The support of claim 76, wherein the biomolecule comprises an RNAi agent.

106. The support of claim 105, wherein the RNAi agent comprises an interfering ribonucleic acid.

107. The support of claim 105, wherein the RNAi agent comprises an oligoribonucleotide present in a duplex structure or a single ribooligonucleotide.

108. The support of claim 105, wherein the RNAi agent is siRNA.

109. The support of claim 105, wherein the RNAi agent is a transcription template of an interfering ribonucleic acid.

110. The support of claim 109, wherein the transcription template is a transcription template of an interfering ribonucleic acid.

111. The support of claim 110, wherein the transcription template is a deoxyribonucleic acid that encodes an interfering RNA.

112. The support of claim 111, wherein the interfering RNA is shRNA.

113. The support of claim 76, wherein the biomolecule comprises a nucleic acid, wherein the nucleic acid is contained in a vector.

114. The support of claim 113, wherein the vector is an expression vector.

115. The support of claim 113, wherein the vector is an episomal vector or a chromosomally integrated vector.

116. The support of claim 76, wherein the biomolecule comprises a protein.

117. The support of claim 76, wherein the biomolecule comprises a virus.

118. The support of claim 76, wherein the support further comprises an enhancer molecule.

119. The support of claim 118, wherein the enhancer molecule comprises an antibody, a peptide, a small molecule, a lectin, a modified polysaccharide, a synthetic composite macromolecule, a functionalized nanostructure, a synthetic polymer, a modified/blocked nucleotides/nucleoside, a modified/blocked amino acid, a fluorophore, a chromophore, a ligand, a chelate, a hapten, a virus, an aptamer, a nucleic acid comprising a ribonucleic acid, a deoxyribonucleic acid, or an oligonucleotide.

120. The support of claim 118, wherein the enhancer molecule comprises a protein.

121. The support of claim 118, wherein the enhancer molecule comprises a RGD peptide.

122. The support of claim 121, wherein the RGD peptide comprises a head-to-tail cyclic pentapeptide, a bicyclic peptide, or a RGD peptide conjugated to a polymer.

123. The support of claim 76, wherein the tie layer is covalently attached to the substrate, the hydrogel layer is covalently attached to the tie layer, and the biomolecule is not covalently attached to the hydrogel layer.

124. The support of claim 76, wherein the substrate is glass, the tie layer is derived from an aminoalkoxysilane, the hydrogel layer is derived from positively-charged dextran, and the biomolecule comprises a nucleic acid, wherein the aminoalkoxysilane is covalently bonded to the glass, the positively-charged dextran is covalently bonded to the aminoalkoxysilane, and the nucleic acid is electrostatically bonded to the positively-charged dextran.

125. The support of claim 76, wherein the support is a slide, a microplate, an array, or a substrate that can support cell growth.

126. A support comprising a substrate, a tie layer, a hydrogel layer, at least one biomolecule, and a cell, wherein the tie layer is covalently bonded to the substrate, the hydrogel layer is attached to the tie layer, the biomolecule is covalently bonded to the hydrogel layer, and the cell is attached to the hydrogel layer.
